(19) United States
(12) Patent Application Publication
(10) Pub. No.: US 2014/0093458 A1
(43) Pub. Date: Apr. 3, 2014

(54) IN VIVO SELECTION OF THERAPEUTICALLY ACTIVE ANTIBODIES

(71) Applicant: Hoffmann-La Roche Inc., Nutley, NJ (US)

(72) Inventors: Michael Dobosz, Penzberg (DE); Werner Scheuer, Penzberg (DE)

(73) Assignee: Hoffmann-La Roche Inc., Nutley, NJ (US)

(21) Appl. No.: 14/018,971

(22) Filed: Sep. 5, 2013

Related U.S. Application Data
(63) Continuation of application No. PCT/EP2012/053801, filed on Mar. 6, 2012.

(30) Foreign Application Priority Data
Mar. 7, 2011 (EP) 11001859.5

(51) Int. Cl. A61K 49/00

(52) U.S. Cl. CPC A61K 49/0058
USPC 424/9.6

(57) ABSTRACT

The present invention relates to non-invasive methods of in vivo selecting an antibody against a desired target on the surface of a mammalian cell of a subject by applying near-infrared fluorescence imaging (NIRF) as well as uses of NIRF in such methods. In addition, kits for the use in these methods comprising a panel of potentially therapeutic effective fluorescence labeled antibodies and means for NIRF imaging are provided.

### RELATED APPLICATIONS

This application is a continuation of International Application No. PCT/EP2012/053801, filed Mar. 6, 2012, the disclosure of which is hereby incorporated by reference in its entirety for all purposes and which claims priority under 35 USC §119 to European Patent Application No. 11001859.5, filed Mar. 7, 2011.

### BACKGROUND

The present invention relates to non-invasive methods of in vivo selecting an antibody against a desired target on the surface of a mammalian cell of a subject by applying near-infrared fluorescence imaging (NIRF) as well as uses of NIRF in such methods. In addition, kits for the use in these methods comprising a panel of potentially therapeutic effective fluorescence labeled antibodies and means for NIRF imaging are provided.

Antibodies account for an increasing portion of marketed human biological therapeutics. Antibodies are used in a whole range of disease fields, such as autoimmunity, cancer, inflammation and infectious disease. Cancer (followed by autoimmunity) is the largest disease area suitable for use of antibodies. The antibody industry is continuously developing new and robust discovery platforms and novel antibody formats, which points to the versatility of antibodies as therapeutic agents. As a consequence, the importance of optimal selection, design, and engineering of antibodies has not only expanded in the past two decades but also is now coming into play as a competitive factor. In fact, design of a therapeutic antibody involves generation, epitope selection, and engineering for optimal efficacy. When antibodies first came into the limelight as therapeutics in the 1980s, the choices for generation, epitope selection, and engineering were rather limited. Since that time however, as more antibodies have been developed and marketed, progress in these areas has been made.

However, though an antibody has been properly designed, it is only half way through before it may be chosen to be a candidate biological therapeutic. Indeed, an antibody must be therapeutically effective. Accordingly, the therapeutic efficacy of an antibody must be tested. Likewise, antibodies for diagnostic applications must be diagnostically effective and must be tested.

Specifically, before therapeutic intervention with a given antibody is initiated, expression of the relevant antigen has to be confirmed. This is commonly accomplished by conventional immunohistochemistry (IHC) or in situ hybridization assays after formalin fixation and paraffin-embedding procedures of explanted tumor tissue. Treatment with a therapeutic antibody is only justified if expression of the relevant tumor-associated target antigen has been confirmed.

In most times, verification of target expression is not performed with the therapeutic antibody, but with an antibody appropriate for IHC. Namely, it is assumed that from the result obtained with an antibody appropriate for IHC, it can be reasonably assumed that an antibody directed against the same target as the antibody appropriate for IHC would be a candidate therapeutic antibody provided that the results obtained with IHC are promising. However, this approach has limitations which may lead to misinterpretations of target confirmation. An antibody applicable for an IHC study has been optimized to fulfil specific criteria required in IHC (e.g. paraffin permeability) and therefore, may have binding characteristics different from the therapeutic antibody. Furthermore, conventional formalin fixation has disadvantages (Chu, Modern Pathol 2005; 18: 850-863). Fixation may modify the surface antigen in such a way that binding of the IHC monoclonal antibody is diminished (or enhanced) which does not reflect the clinical situation. In addition, formalin fixed paraffin embedded tissue slides have to be processed in special ways to get optimal staining with an IHC antibody (e.g. antigen retrieval by heating or incubation with special enzymes).

Another technique to select therapeutically active antibodies is selection by kinetic analysis (Esaki, Biacore Journal 2002; 2: 7-8). Like IHC, also this method is made in vitro under artificial conditions. However, so far and in the absence of alternatives, this method is used for the selection of therapeutically active antibodies.

Yet, prior art techniques for the selection of a therapeutically effective or even the potentially most therapeutic antibody suffer from the deficiency that in vivo parameters are not taken into account. For example, tumor-associated surface antigens can be modulated (overexpressed, internalized or shedded) during tumor growth and metastatic spread and, thus, an in vitro therapeutically promising antibody may not show the same efficacy in vivo.

In theory, one may thus think of the direct injection of a purified pool of antibodies to patients. The rationale would be that among a pool of antibodies a suitable candidate would be present. However, this theoretical thought cannot be materialized in reality. Indeed, it would neither be possible nor desirable. This is especially true in the case of long-term therapies for at least two reasons. One is the possibility of inducing anti-idiotypic immune responses as a result of repeated injection of massive doses of antibody, let alone any other potential risks for the health of the patient. The other is the high cost of antibodies and the fact that one would never know which antibody of a pool of antibodies is the therapeutically most effective one.

Thus, since antibodies are the second largest medicine category after vaccines and antibody-based therapies are expensive and should thus be optimized for patients in terms of therapeutic efficacy, a technique which allows verification of target expression and demonstration of binding of the therapeutic antibody simultaneously in vivo at the time point when therapy will be initiated, would be highly desirable in order to select among a panel of potentially therapeutic effective antibodies a candidate antibody that is potentially therapeutically most active. In addition, it would be highly desirable if such a technique would reflect in vivo conditions.

Hence, the technical problem of the present invention is to comply with the needs described above.

The present invention addresses these needs and thus provides as a solution to the technical problem the embodiments concerning means, for example, tools and kits as well as methods and uses applying these means for in vivo selecting an antibody against a desired target on the surface of a mammalian cell (preferably a malignant cell) that is a candidate for the therapeutically most effective antibody out of a panel of potentially therapeutic effective antibodies. These embodiments are characterized and described herein, illustrated in the Examples, and reflected in the claims.

The aspects of the present invention can be summarized in the following aspects/items:

(1) A non-invasive method of in vivo selecting an antibody against a desired target on the surface of a mammalian cell of a subject that is a candidate for the therapeutically most effective antibody out of a panel of potentially therapeutic effective antibodies (binding the same desired target) each of which is fluorescence labeled comprising
(a) detecting each of the fluorescence labeled potentially therapeutic effective antibodies of said panel after binding to its target in a subject by near-infrared fluorescence imaging (NIRF); and
(b) selecting the antibody which shows after binding to its target the highest fluorescence signals.

(2) The method of aspect 1, wherein said panel of antibodies binding the same desired target is fluorescence labeled with just one sort of fluorescent label.

(3) The method of any of the preceding aspects, wherein said mammalian cell is a malignant cell.

(4) The method of any of the preceding aspects, wherein said panel of potentially therapeutic effective antibodies comprises antibodies intended to be used in therapy of said subject.

(5) The method of any of the preceding aspects, wherein the subject is to be administered the fluorescence labelled potentially therapeutic antibody prior to detecting said antibody.

(6) The method of any of the preceding aspects, wherein said subject is a mammal.

(7) The method of any of the preceding aspects, wherein said subject comprises a tumor which comprises malignant/cancerous cells having said desired target on their surface.

(8) The method of aspect 6, wherein said tumor is a xenograft, preferably a human xenograft.

(9) The method of any of the preceding aspects, wherein the target is a protein having an extracellular portion.

(10) The method of any of the preceding aspects, wherein near-infrared fluorescence imaging includes fluorescence reflectance imaging (FRI) or fluorescence-mediated tomography (FMT).

(11) The method of any of the preceding aspects which is for in vivo selecting an antibody which is most appropriate for therapy.

(12) Use of the method of any of the preceding aspects for in vivo selecting an antibody against a desired target on the surface of a mammalian cell that is a candidate for the therapeutically most effective antibody out of a panel of potentially therapeutic effective antibodies.

(13) Use of near-infrared fluorescence imaging (NIRF) in a method for in vivo selecting an antibody against a desired target on the surface of a mammalian cell that is a candidate for the therapeutically most effective antibody out of a panel of potentially therapeutic effective antibodies (binding the same desired target) each of which is fluorescence labeled comprising
(a) detecting each of the fluorescence labeled potentially therapeutic effective antibodies of said panel after binding to its target in a subject by near-infrared fluorescence imaging (NIRF),
(b) selecting the antibody which shows after binding to its target the highest fluorescence signals.

(14) Use of aspect 13, wherein said panel of antibodies binding the same desired target is fluorescence labeled with just one sort of fluorescent label.

(15) An antibody selected by the method of any of the preceding aspects for use in the treatment of cancer.

(16) Use of an antibody selected by the method of any of the preceding aspects for the preparation of a pharmaceutical composition for the treatment of cancer.

(17) A kit for use in any of the methods defined in the preceding aspects comprising a panel of potentially therapeutic effective fluorescence labeled antibodies and means for near-infrared fluorescence imaging to detect said antibody in a subject.

It must be noted that as used herein, the singular forms “a”, “an”, and “the”, include plural references unless the context clearly indicates otherwise. Thus, for example, reference to “a reagent” includes one or more of such different reagents and reference to “the method” includes reference to equivalent steps and methods known to those of ordinary skill in the art that could be modified or substituted for the methods described herein.

All publications and patents cited in this disclosure are incorporated by reference in their entirety. To the extent the material incorporated by reference contradicts or is inconsistent with this specification, the specification will supersede any such material.

Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the present invention.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or sometimes when used herein with the term “having”.

When used herein “consisting of” excludes any element, step, or ingredient not specified in the claim element. When used herein, “consisting essentially of” does not exclude materials or steps that do not materially affect the basic and novel characteristics of the claim. In each instance herein any of the terms “comprising”, “consisting essentially of” and “consisting of” may be replaced with either of the other two terms.

As used herein, the conjunctive term “and/or” between multiple recited elements is understood as encompassing both individual and combined options. For instance, where two elements are conjoined by “and/or”, a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or” as used herein.

As described herein, “preferred embodiment” means “preferred embodiment of the present invention”. Likewise, as described herein, “various embodiments” and “another embodiment” means “various embodiments of the present invention” and “another embodiment of the present invention”, respectively.

Several documents are cited throughout the text of this specification. Each of the documents cited herein (including all patents, patent applications, scientific publications, manufacturer's specifications, instructions, etc.), whether supra or infra, are hereby incorporated by reference in their entirety. Nothing herein is to be construed as an admission that the invention is not entitled to antedate such disclosure by virtue of prior invention.

The inventors of the present application with the aim of selecting the therapeutically most effective antibody out of a panel of potentially therapeutic effective antibodies developed the methods of the present invention described herein which allow verification of target expression and demonstration of the binding of the therapeutic antibody simultaneously in vivo at the time point when therapy will be initiated.

The inventors labelled therapeutic antibodies with an organic fluorophore and injected it intravenously (i.v.) in mice having human xenografts. They demonstrated the utility of near infrared fluorescence (NIRF) to verify target expression and binding to tumor tissue. After a single i.v. injection of 50 microgram of Cy5 labelled Trastuzumab antibody (Tradename: Herceptin) or Pertuzumab antibody (Tradename: Omnitarg) in mice (which carry a Her2 positive tumor that is recognized by Trastuzumab and Pertuzumab) a strong fluorescence signal is detectable in the tumor area after 24 to 48 hours. Subsequent analysis of explanted tumor tissue reveals that the Her2 specific antibodies bind only to tumor cells, but not to murine tissue. In contrast, no fluorescence signal is generated with a control antibody Xolair which is directed to human IgE (FIG. 1). This approach is superior to conventional detection of target antigens by classical IHC because i) the relevant target is in its “natural environment” ii) modulation of target expression during the primary tumor growth and metastatic spread can be detected iii) explantation of tumor tissue and fixation is be performed after the antibody has bind to the relevant target iv) differences in binding characteristics of therapeutic antibody and IHC antibody become probably obsolete or may only be carried out for confirmation.

In their studies the present inventors observed that antibodies that showed good performance in in vitro tests such as IHC, did not necessarily have sufficient therapeutic activity. Likewise, antibodies that showed good performance in near-infrared imaging, had surprisingly sufficient therapeutic activity. From these observations the inventors concluded that the so far applied techniques for selecting the therapeutically most effective antibody out of a panel of potentially therapeutic effective antibodies may either produce false-positive or false-negative results and, thus, are not satisfying to reliably select a promising candidate antibody for an antibody-based therapy in, for example, treating cancer. However, the methods of the present invention take into account the expression level of the desired target on the surface of a mammalian cell, target internalization and/or a potential shedding of the target.

Specifically, the inventors labelled potentially therapeutic effective antibodies against Cyr61 with the organic fluorophore Cy5 and injected them (each individually) intravenously (i.v.) in mice having human xenografts. To this end, the present inventors labeled four different therapeutic antibodies (MOR06395, MOR06396, MOR06434, MOR06420) against the tumor antigen Cyr61 with the organic fluorophore Cy5. Each of the labelled antibodies was injected in three s.c. tumor bearing SCID-beige mice (human pancreatic xenograft). After a single i.v. injection of 50 microgram of the labelled anti-Cyr61 mAb, the fluorescence signal was imaged in the tumor area after 24 to 48 hours (FIG. 2). The signals of all in vivo images were normalized, converted into pseudocolor and the intensities in the tumor area were analysed. Comparing the signal intensities of the four different antibodies with each other, mAb MOR06420 shows the highest signal intensity in the tumor area. The results from in vivo imaging were confirmed by histological ex vivo analysis. For this, the tumors were explanted, fixed in formalin and embedded in paraffin. In the sliced and DAPI stained tumor tissue, antibody MOR06420 shows also the brightest signal intensity (FIG. 3). From the in vivo and ex vivo results, mAb MOR06420 can be prioritized as the antibody with the best binding efficacy.

Therefore, the antibody MOR06420 was assumed to show the best therapeutic efficacy. To confirm this hypothesis, the four different therapeutic antibodies against Cyr61 were tested in a preclinical study. The s.c. Panc-1 bearing SCID-beige mice were divided in five groups with ten mice each. The untreated vehicle group was injected i.p. with Histidine buffer twice a week. Each of the four treatment groups was injected i.p. with an anti-Cyr61 antibody concentration of 20 mg/kg twice a week. The tumor volume of all animals was measured over the time by calliper (FIG. 4). Indeed, the anti-Cyr61 monoclonal antibody MOR06420 appears to possess the most potent antitumor activity in Panc-1 xenografts, with a tumor growth inhibition of 68%.

Accordingly, in a first aspect the present invention relates to a non-invasive method of in vivo selecting an antibody against a desired target on the surface of a mammalian cell of a subject that is a candidate for the therapeutically most effective antibody out of a panel of potentially therapeutic effective antibodies each of which is fluorescence labelled comprising
(a) detecting each of the fluorescence labelled potentially therapeutic effective antibodies of said panel after binding to its target in a subject by near-infrared fluorescence imaging (NIRF); and
(b) selecting the antibody which shows after binding to its target the highest fluorescence signals.

Accordingly and in essence, the method of the present invention is for in vivo selecting an antibody which is most appropriate for therapy. It is likewise for optimizing a therapeutically effective antibody. Also, it can be applied for personalizing an antibody-based therapy in that out of a panel of potentially therapeutic antibodies the therapeutically most effective antibody for a subject (i.e. an individual) is selected. Thus, the present invention enables the tailoring of an antibody-based therapy in that the therapeutically most effective antibody is individually selected by performing the methods of the present invention.

Further, the methods of the present invention allow the testing as to whether a panel of potentially therapeutic antibodies comprising at least two, three, four, five, six, seven, eight, nine, ten or more antibodies is suitable for a subject. Specifically, the methods described herein allow the simultaneous testing of two, three, four, five, six, seven, eight, nine, ten or more antibodies directed against a desired target in vivo in a subject, thereby allowing the determination as to whether two, three, four, five, six, seven, eight, nine, ten or more antibodies would adversely interfere each other (for example, the antibodies may compete for the same epitope or would bar each other from binding due to steric hindrance). This is an important aspect since it may be desirable to administer two or more antibodies directed against the same desired target to a subject in order to increase the efficacy of an antibody-based therapy.

Likewise, the methods of the present invention allow the testing as to whether antibodies against two, three, four, five, six, seven, eight, nine, ten or more targets would interfere with each other in a multi-antibody based therapy. These aspects of the present invention aid in assessing potential adverse effects for a patient.

Also, the methods of the present invention allow the determination of the specificity of an antibody in vivo since its binding to the desired target and its binding to non-desired targets (i.e. non-specific binding) can be monitored in vivo.

The methods and uses described herein allow real-time in vivo selection of an antibody against a desired target on the surface of a mammalian cell that is a candidate for the therapeutically most effective antibody out of a panel of potentially therapeutic effective antibodies, i.e., no in vitro step in selecting such an antibody is required and/or no delay in generating and collecting data such as developing pictures or the like will occur. Accordingly, any change in the performance of the antibody when selecting a candidate for the therapeutically most effective antibody out of a panel of potentially therapeutic effective antibodies may be directly (online) visualized.

An advantage of the method of the present invention is thus that it allows imaging under real time and the performance of potentially therapeutic antibodies in vivo so as to allow the selection of the therapeutically most effective antibody out of a panel of potentially therapeutic effective antibodies. Accordingly, the method of the present invention allows verification of target expression and demonstration of binding of the therapeutic antibody simultaneously in vivo at the time point when therapy will be initiated.

In fact, the inventors found that antibodies which showed good results in in vitro test such as IHC which is a standard for selecting therapeutically effective antibodies do not show the promising therapeutic efficacy in vivo. In particular, the inventors applied NIRF to monitor the in vivo performance of these promising antibodies and found that these antibodies fail to efficiently bind the target.

Likewise, the inventors found surprisingly that antibodies which failed in in vitro tests are promising candidates for therapeutically effective antibodies when they monitored the performance of these “failed” antibodies by applying NIRF.

Hence, the inventors concluded that the commonly applied in vitro techniques for selecting candidates for therapeutically effective antibodies out of a panel of potentially therapeutic antibodies are not reliable, while such candidate antibodies can be more reliably identified by applying a method which monitors the performance of antibodies under real-time conditions in vivo, i.e. by applying the methods of the present invention.

The method of the present invention is also applicable to select a candidate for the diagnostically most effective antibody out of a panel of potentially diagnostic effective antibodies each of which is fluorescence labeled. Accordingly, as far as a diagnostic aspect is concerned, in all embodiments described herein, the term “therapeutically” or “therapeutic” or any other grammatical form thereof is to be understood to mean “diagnostically” or “diagnostic” and any grammatical form thereof.

The methods of the present invention are non-invasive. “Non-invasive” means that no break in the skin of a subject is created, for example, an incision, and there is no contact with the mucosa, or skin break, or internal body cavity beyond a natural or artificial body orifice in order to perform the methods and uses of the present invention.

The term “in vivo” includes selecting a candidate for the therapeutically most effective antibody out of a panel of potentially therapeutic effective antibodies antibody under real-time conditions. It thus refers to localizing and/or detecting a potentially therapeutic effective antibody using an imaging or scanning technology in a living subject.

Accordingly, the performance of an antibody can be monitored in a living organism. Hence, the methods of the present invention are not carried out in a partial or dead organism. The in vivo selection of an antibody in a living subject thus reflects the scenario that occurs/is present in a living subject; thereby it takes into account the naturally-occurring conditions in a subject.

For example, the age, body weight, general health, sex, diet, drug interaction and the severity of the condition may influence the therapeutic activity of an antibody.

The selection method of the present invention is done “in vivo”. Accordingly, the methods and uses of the present invention which apply in vivo imaging are not carried out in a partial or dead organism.

Therefore, the methods of the present invention allow to select a candidate antibody that is most effective for an individual subject, i.e. the methods allow a personalized/individualized therapy for a subject, since the in vivo conditions, though subjects may suffer from the same disease, may be different and, thus, an antibody which is therapeutically most effective for one subject may not necessarily be as effective in another subject and vice versa. For example, the target antigen may be differently expressed in terms of its expression level in a subject, may be buried, unusually glycosylated, etc., thereby creating differences among subjects. However, the methods of the present invention overcome these potential problems, thereby making available the personalization of an antibody-based therapy.

A “panel” of potentially therapeutic effective antibodies may include at least 1, 2, 3, 4, 5, 6, 7, 8, 9, 1.0, 11, 12, 13, 14, 15, 16, 17, 18, 19, 20, 21, 22, 23, 24, 25, 26, 27, 28, 29, 30, 31, 32, 33, 34, 35, 40, 45, 50, 55, 60, 65, 70, 75, 80, 85, 90, 95, 100, 120, 140, 150, 160, 180, 200, 220, 240, 250, 260, 280, 300, 400, 500, 600, 700, 800, 900 or 1000 antibodies. The antibodies comprised by the panel of potentially therapeutic effective antibodies are fluorescence labeled, i.e., each of the antibodies comprised by said panel is fluorescence labeled.

Without being bound by theory, it is envisaged that the antibodies comprised by the panel, though binding the same desired target, are different from each other. For example, they may bind to different epitopes of said target or may bind with different affinity to said target.

In a preferred embodiment, the panel of potentially therapeutic effective antibodies comprises antibodies intended to be used in therapy of the subject.

A subject when used herein includes mammalian and non-mammalian subjects, with mammalian subjects being preferred. “Mammal” for purposes of the invention refers to any animal classified as a mammal, including human, domestic and farm animals, nonhuman primates, and any other animal that has mammary tissue. A mammal includes human, rodents such as mouse, rat, guinea pig, rabbit, dog, cat, horse, camel, pig, cow, goat, chimpanzee etc., with human being preferred. A subject also includes human and veterinary patients.

The subject preferably comprises a tumor which comprises malignant cells having the desired target as described herein on the surface of said malignant cells.

A non-mammalian subject is, for example, a chicken, a fowl, a duck or a zebra fish. It is also envisaged that the subject of the present invention comprises a tumor.

The subject also includes non-human subjects. Non-human subjects may represent a model of a particular disease or disorder. It is also envisaged that the non-human subject of the present invention comprises a xenograft, preferably a human xenograft, more preferably a human tumor.

This aspect contemplates a pre-clinical model for in vivo selecting an antibody against a desired target on the surface of a mammalian cell that is a candidate for the therapeutically most effective antibody out of a panel of potentially therapeutic effective antibodies.

In some embodiments, the subject has a solid tumor. Solid tumors may be benign (not cancer), or malignant (cancer).

In some embodiments, the subject already comprises one or more of the antibodies described herein. Put it differently, said one or more antibodies is administered to the subject prior to performing the methods and uses of the present invention. Thus, the subject is, so to say, pre-treated with said one or more antibodies. However, the antibody is preferably not yet therapeutically effective, since it is administered in a non-therapeutically amount.

In a preferred aspect of the methods of the invention the solid tumor of the subject is a xenograft, preferably a human xenograft, more preferably a human tumor. Typically, the skilled person would use the same xenograft for all antibodies if a panel of antibodies should be tested for the therapeutically most effective antibody against a desired target (e.g., in one case a Panc-1 xenograft for the comparison of all antibodies out of a panel of CYR61 antibodies, in another case a CALU-3 xenograft for the comparison of all antibodies out of a panel of HER3 antibodies).

The term “solid tumor” when used herein refers to tumors elected from the group of gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, testicular cancer, esophageal cancer, tumors of the biliary tract, as well as head and neck cancer, preferably breast cancer.

For the purpose of the present invention, a solid tumor has a size of at least 1 mm, preferably 2 mm. At this size, a solid tumor in order to proliferate and to survive depends on an adequate supply of growth factors and the removal of toxic molecules as well as on a sufficient oxygen supply. In solid tissues, oxygen, for example, can diffuse radially from capillaries for only about 150 to 200 μm and, thus, the tumor depends on the supply of oxygen by blood vessels and, thus, initiates angiogenesis.

For example, a solid tumor refers to a tumor selected from the group of gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, testicular cancer, esophagael cancer, tumors of the biliary tract, as well as head and neck cancer.

When used herein the term “cancer” refers to a medical condition that is characterized by the growth of a tumor. It is thus the physiological condition in a subject that is typically characterized by unregulated cell growth.

A mammalian cell is a cell of a mammal as described herein. It includes all types of cells, for example, cells of organs, muscle cells, nerve cell, cells of the immune system (lymphocytes including PBMCs, B-cells, T-cells, NK cells, macrophages), eosinophils, neutrophils, basophils. In preferred embodiments, said mammalian cell is a malignant cell.

“Malignant” describes a cell that contributes to a progressively worsening disease, in particular growth of a tumor or the diseases described herein. The term is most familiar as a description of cancer including growth of tumor. Malignant cells are not self-limited in their growth, are capable of invading into adjacent tissues, and may be capable of spreading to distant tissues (metastasizing). Malignant when used herein is synonymous with cancerous.

“Detecting” when used herein refers to any technique or process in connection with near-infrared fluorescence imaging including fluorescence imaging and/or scanning technology used to detect signals emitted from a fluorescence label, for example, from a fluorescence label of the antibody described herein. It includes creating images of the region of the subject's body (or parts thereof), where the fluorescence signal is expected to be localizable. For example, if a fluorescence labelled antibody is expected to bind to the surface of a mammalian cell of the liver, images are created from the region of the body where the liver is located.

Near-infrared fluorescence imaging which is preferred when detecting the fluorescence labelled antibodies applied in the methods of the present invention is a spectroscopic method which uses the near infrared region of the electromagnetic spectrum. In a preferred embodiment, near-infrared fluorescence imaging includes fluorescence reflectance imaging (FRI) or fluorescence-mediated tomography (FMT).

A “region” includes any part of the subject's body, any organ of the subject and the surface of the subject's body including skin, muscles and bones. “Organ” includes one or more organs selected from the digestive system (including salivary glands, esophagus, stomach, liver, gallbladder, pancreas, intestines, rectum and anus); endocrine system (including endocrine glands such as the hypothalamus, pituitary or pituitary gland, pineal body or pineal gland, thyroid, parathyroids and adrenals, i.e., adrenal glands); integumentary system (including skin, hair and nails); lymphatic system (including lymphatic system, lymph nodes, tonsils, adenoids, thymus and spleen); muscular system; nervous system (including brain, spinal cord, peripheral nerves and nerves); reproductive system (including ovaries, fallopian tubes, uterus, vagina, mammary glands, testes, vas deferens, seminal vesicles, prostate and penis); respiratory system (including the pharynx, larynx, trachea, bronchi, lungs and diaphragm); skeletal system (including bones, cartilage, ligaments and tendons); and the urinary system (including kidneys, ureters, bladder and urethra involved in fluid balance, electrolyte balance and excretion of urine).

Likewise, it is envisaged that images are created from the region as described herein and/or from further regions of the subject. Specifically, it may be of interest to create images from the entire body of the subject to localize the fluorescence labelled antibody. For example, it may be of interest to monitor the binding and/or presence of the antibody in the blood to, for example, determine the binding specificity of the antibody and/or its distribution, half-time, etc. in order to aid in the selection of a candidate for the therapeutically most effective antibody out of a panel of potentially therapeutic effective antibodies.

“From further regions of the subject” means further defined or discrete parts, or regions of the subject, which might be of interest for any kind of measurement. For example, it is envisaged that the organ distribution and/or accumulation and/or secretion (determination of the secretion pathway) and/or metabolism (for example the generation of metabolites of a drug) of a fluorescence labeled antibody is to be detected and/or evaluated, which will for example aid in the determination of a candidate for the therapeutically most effective antibody out of a panel of potentially therapeutic effective antibodies. “Further regions” therefore comprises for example a part of an organ, an organ, blood vessel networks or nervous cell system of the subject.

The created images can then be qualitatively and/or quantitatively analyzed. In the context of the method of the present invention, the created images are analyzed to determine the fluorescent signals from each of the fluorescence labelled antibodies from which a candidate for the therapeutically most effective antibody out of a panel of potentially therapeutic effective antibodies is to be selected. Specifically, the antibody which shows after binding to its target the highest fluorescence signals is a candidate for the therapeutically most effective antibody out of a panel of potentially therapeutic effective antibodies. More specifically, the height (i.e. quantitative amount) of the fluorescence signals of each of the antibodies is determined and compared relatively to each other, i.e., the height of the signal of all tested antibodies is determined and compared to each other, whereby the highest signal can be determined. Optionally, a background value (i.e. the height of the fluorescence signal emitted from the fluorescence labelled antibody when it is located elsewhere in the body, but not at the desired target) may be subtracted from the height of the fluorescence signal of each antibody (i.e. each fluorescence signal is normalize) before the highest signal is determined.

A qualitative analysis of the created images is also envisaged, but less preferred, since the method of the present invention is for selecting a candidate for the therapeutically most effective antibody, whereby such a candidate can be selected on the basis of the highest fluorescence signals. In fact, the inventors observed that the antibody which shows the highest fluorescence signal after binding to its target is such a candidate antibody.

As used herein, the term “binds” in all its grammatical forms when used in connection with the interaction between a desired target and the antibody to be selected in accordance with the present invention indicates that the antibody's epitope binding domain (i.e. the CDRs of the variable light and heavy chains (in case of domain antibodies only the CDRs of either the light or heavy chain), optionally in combination with one or more amino acids from the framework regions) associates with (e.g., interacts with or complexes with) the target to an antibody-antigen complex in a statistically significant degree as compared to association with proteins generally (i.e., non-specific binding). The term “epitope binding domain” is also understood to refer to a domain that has a statistically significant association or binding with a target.

Preferably, the method of the present invention aids in the in vivo selection of an antibody against a desired target on the surface of a mammalian cell that is a candidate for the therapeutically most effective antibody out of a panel of potentially therapeutic effective antibodies. The term “aiding” is used to indicate that the method according to the present invention will (together with other methods and/or variables, e.g., methods for selecting a therapeutically effective antibody or clinical parameters) aid the skilled artisan to select an antibody against a desired target on the surface of a malignant/cancerous cell that is a candidate for the therapeutically most effective antibody out of a panel of potentially therapeutic effective antibodies. Put it differently, said other methods will help to confirm the selection of the candidate for the therapeutically most effective antibody out of a panel of potentially therapeutic effective antibodies. Examples of said other methods are immunochemistry, immunohistochemistry, histological techniques, for example, issue staining, or mRNA/PCR applications.

Specifically, for tissue staining, tumors of a subject, preferably a test animal such as a mouse, rat or monkey are explanted, sections are prepared, fixed, embedded in paraffin and stained with, for example, hematoxylin-eosin (HE). So-stained tumor sections can be imaged ex vivo by, for example, the Nuance® system. As an example for tumor explanation, test animals are sacrificed and tumors are explanted according to committed guidelines (GVSolas, Felasa, TierschG). After sacrificing the animals, tumors are explanted using, for example, a scalpel and transferred into an embedding cassette or flash frozen in liquid nitrogen with isopentan for immunohistochemical applications and without isopentan for mRNA/PCR applications.

As an example for fixation, explanted tumors are enclosed in an embedding cassette and are incubated under continuous agitation in formalin for preferably approximately 24 hours. Thereafter, formalin is discarded and tumors washed with dest. water followed by dehydration of the tumors and penetration of paraffin. All the incubation steps are carried out using the Tissue Tek® VIP Vacuum Infiltration Processor. After paraffin penetration, tumors are embedded in liquid paraffin with the Tissue Tek® Paraffin embedding station to a final histological block. Paraffin sections in a range of 2-8 μm of thickness are obtained from these blocks using a microtome. They are cut, taken up on glass slides, air dried over night at 37° C. followed by de-paraffinization and HE-staining Slides are examined with the Nuance system.

As regards immunohistochemistry, an unlabeled antibody is parenterally injected, preferably intravenously, tumors are explanted, preferably at around 24 hours post-injection of the antibody, flash frozen in, for example, liquid nitrogen and isopentan and sectioned. Unlabeled antibody is identified by using a secondary antibody directed against the Ig subtype of the unlabeled antibody, for example, goat anti-mouse IgG.

For Immunohistochemistry applications, tumors are flash frozen immediately after excision in Isopentan and liquid nitrogen and held at −20° C. until sectioning. For example, 10 μm tumor cryosections are cut at the optimum cutting temperature of −18 C with the Cryostat (2800 Frigocut), fixed with acetone for one minute, air dried and used for Immunohistochemistry application.

With respect to mRNA techniques, tumors are explanted and flash frozen in, for example, liquid nitrogen (without isopentan). Tumors are lysed, homogenized and RNA is prepared according to methods commonly known in the art. Afterwards, cDNA is prepared and analyzed qualitatively and/or quantitatively for the expression of a gene of interest, for example, that encoding the target on the surface of a mammalian cell against which an antibody is selected in accordance with the teaching of the present invention. As a loading control in a quantitative expression analysis a typical housekeeping gene such as β-actin or GAPDH can be used.

A yet further “other method” is the selection by kinetic analysis by using Biacore. Specifically, the antibody with the highest affinity to its target is selected by Biacore, whereby it is assumed that such an antibody could be therapeutically most effective.

These other method will be one of the components taken into consideration by the skilled artisan helping i.e. aiding him to select an antibody against a desired target on the surface of a malignant/cancerous cell that is a candidate for the therapeutically most effective antibody out of a panel of potentially therapeutic effective antibodies.

The term “therapeutically effective” or “therapeutic efficacy” or any other grammatical form thereof refers to the capability of an antibody to “treat” a disease or disorder in a subject. In the case of cancer, the therapeutically effective amount of the antibody may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer.

The therapeutic effect which an antibody may have can be detected by all established methods and approaches which will indicate a therapeutic effect. It is, for example, envisaged that the therapeutic effect is detected by way of surgical resection or biopsy of an affected tissue/organ which is subsequently analyzed by way of immunohistochemical (IHC) or comparable immunological techniques. Alternatively it is also envisaged that the tumor markers in the serum of the patient (if present) are detected in order to diagnose whether the therapeutic approach is already effective or not. Additionally or alternatively it is also possible to evaluate the general appearance of the respective patient (fitness, well-being, decrease of tumor-mediated ailment etc.) which will also aid the skilled practitioner to evaluate whether a therapeutic effect is already there. The skilled person is aware of numerous other ways which will enable him or her to observe a therapeutic effect. For cancer therapy, therapeutic efficacy can be measured, for example, by assessing the time to disease progression (TTP) and/or determining the response rate (RR).

A “potentially therapeutic effective” antibody is an antibody that has a likelihood or probability to be therapeutically effective. Accordingly, the term “potential” when used in the context of the antibodies that can be selected in accordance with the methods of the present invention means that—though such an antibody is deemed to have a therapeutic effect—said antibody does not necessarily have to be therapeutically effective. However, the methods of the present invention aid in selecting a candidate for the therapeutically most effective antibody out of a panel of potentially therapeutic effective antibodies.

A “candidate for the therapeutically most effective antibody” is an antibody that is a prospective antibody that may be useful in an antibody-based therapy. Such a candidate shows preferably improved properties in vivo (such as binding, target recognition, affinity) in comparison to other potentially therapeutic effective antibodies. Preferably, said candidate is in accordance with the methods of the present invention selected out of a panel of potentially therapeutic effective antibodies on the basis that it shows after binding to its target on the surface of a mammalian cell in vivo the highest fluorescence signals.

An imaging system useful in the practice of this invention typically includes three basic components: (1) excitation light, (2) a means for separating or distinguishing excitation light and emission light (preferably a software and/or hardware filter(s) which might be fitted to the excitation light and/or to the detection system), and (3) a detection system for receiving the light emitted from at least one fluorescent label and/or from the fluorescent entity and/or from the fluorescent analyte of the invention (optical detector). It is envisaged that the light source (excitation means) may optionally (i) comprise a pre-determined or tunable filter. The light source can be a suitably filtered white light, i.e., bandpass light from a broadband source. For example, light from a 150-watt halogen lamp can be passed through a suitable bandpass filter. In some embodiments, the light source is a laser. See, e.g., Boas et al., 1994, Proc. Natl. Acad. Sci. USA 91:4887-4891; Ntziachristos et al., 2000, Proc. Natl. Acad. Sci. USA 97:2767-2772; Alexander, 1991, J. Clin. Laser Med. Surg. 9:416-418. Information on near infrared lasers for imaging can be found at http://www.imds.com and various other well-known sources. A high pass or bandpass filter (e.g. 700 nm) can be used to separate optical emissions (emission light) from excitation light. Any suitable light detection/image recording component (an optical detector), e.g., charge coupled device (CCD) systems, a photodiode, a photoconductive cell, a complementary metal oxide semiconductor (CMOS) or photomultiplier tubes can be used in the invention. Said components are explained in more detail herein below. The choice of light detection/image recording will depend on factors including type of light gathering/image forming component being used. Selecting suitable components, assembling them into an imaging system of the invention, and operating the system is within ordinary skill in the art.

The in vivo imaging techniques applied in the present invention preferably include the FMT technology (fluorescence molecular tomography), a laser based three-dimensional imaging system, provides non-invasive, whole body, deep tissue imaging in small animal models and generates 3D reconstruction of fluorescence sources and/or allows measurement of fluorescence concentrations of fluorescence labeled structures due to binding of the fluorescence labeled antibody to its desired target as described herein. The system allows determining the real distribution of the fluorescence labeled antibody in a planar, two-dimensional imaging mode and/or 3D imaging studies with the FMT system allow improved assessment of the antibody distribution in the subject's body) Therefore, quantification of fluorescence signal intensities can be analyzed in a more realistic approach.

An example of a near-infra red fluorescence imaging system is the MAESTRO system described in the appended Examples. Accordingly, the MAESTRO system is a preferred system that may be applied in the kits, methods and uses of the present invention.

The MAESTRO system is a planar fluorescence-reflecting-imaging system that allows a noninvasive in vivo fluorescence measurement. In this multispectral analysis, a series of images are captured, at specific wavelengths. The range of wavelengths captured should cover the expected spectral emission range of the label present in the specimen. The result will be a series of images called “image cube” and it is the data within this series of images that is used to define the individual spectra of both auto-fluorescence and specific labels. Many labels of biological interest have emission spectra that are so similar that separation using expensive narrow band filters is difficult or impossible. A single long pass emission filter replaces a large collection of emission filters. In addition to the natural auto-fluorescence of the skin, fur, sebaceous glands, there is also distinct auto-fluorescence from commensal organisms (fungi, mites, etc.) and ingested food (chlorophyll). Multispectral analysis is able to separate all of these signals from the specific label applied to the specimen through the mathematically disentanglement of the linear signal mixture (unmixing) of the emitted fluorescent lights as long as the emission spectrum of the desired signal and of the auto-fluorescence are known.

Measurement with the MAESTRO system works as follows: The illumination module is equipped with a xenon lamp (Cermax) that excites white light. Through a downstream connected excitation filter (chosen by the experimenter), the light is delimitated to a, for the experiment, desired wavelength range and conducted via an optical fiber into the imaging module. In here, the restricted light is partitioned into four optical fibers that illuminate the anesthetized test animal. The MAESTRO system chooses the optimal exposure time automatically, so that there is no risk of overexposure. The emitted fluorescence light of the activated fluorescent probe is selected with an emission filter (see Table 1) and conducted through a liquid crystal (LC) to a high sensitive, cooled CCD-camera. The liquid crystal enables the camera a selective picture recording of a specific wavelength. The wavelength measurement range depends on the selected filter set (blue, green, yellow, red, deep red, NIR) and pictures are recorded in steps of 10 nm. The spectral information of each single picture is combined in one “picture package” that is called “image cube”.

TABLE 1
Maestro filter sets.

| Maestro | | Excitation | Emission | Acquisition |
|---|---|---|---|---|
| Filter Set | Part # | Filter | Filter | Settings* |
| Blue | M-MSI-FLTR- | 445 to | 515 nm | 500 to 720 in |
| | BLUE | 490 nmm | longpass | 10 nm steps |
| Green | M-MSI-FLTR- | 503 to | 580 nm | 550 to 800 in |
| | GREEN | 555 nm | longpass | 10 nm steps |
| Yellow | M-MSI-FLTR- | 575 to | 645 nm | 630 to 850 in |
| | YELLOW | 605 nm | longpass | 10 nm steps |
| Red | M-MSI-FLTR- | 615 to | 700 nm | 680 to 950 in |
| | RED | 665 nm | longpass | 10 nm steps |
| Deep Red | M-MSI-FLTR- | 671 to | 750 nm | 730 to 950 in |
| | DEEP-RED | 705 nm | longpass | 10 nm steps |
| NIR | M-MSI-FLTR- | 710 to | 800 nm | 780 to 950 in |
| | NIR | 760 nm | longpass | 10 nm steps |

The analysis with the MAESTRO system works as follows:

Each recording compose of 12 bit black-and-white pictures that can be illustrated in 4096 different gray scales and therefore it is possible to discriminate between smallest differences in emission intensities. In contrast, the human eye is able to distinguish between 30-35 grey scales. Those values for the emission intensities (grey scales) are plotted against the wavelength range and as a result, we obtain the emission spectra of each probe and the tissue auto-fluorescence. The software subdivides the three fundamental colors (red, green, blue) to the wavelength range used for the imaging cube whereby the black-and-white pictures turn into colored image. Out of these acquired multispectral information the system is able to differentiate between injected probes and auto-fluorescence of any source. The program is using a spectral library, where the single spectra of each pure probe and the spectra acquired by imaging the study animals (Balbc/nude or Scid Beige mice) without any injection (mouse auto-fluorescence). By knowing the exact spectra of the pure imaging and of the auto-fluorescence, the system is able to filter the whole image for the desired spectra and assign a color to each of them. The originated image (unmixed composite image) shows the present spectra in different colors. To visualize the intensity distribution of the probe signal, it is possible to illustrate the signal in false colors, whereas low intensities are blue and regions of high intensities are red. Besides that, one can define a detection limit for the signal intensity of the probe, which allows reducing the signal of circulating probes and unspecific bindings.

Comparison and quantification with the MAESTRO system works as follows: The MAESTRO's ability to compare fluorophore regions of an image makes it easy to compare the tumor fluorescent signal intensities during therapy. The program provides tools for the comparison of different signal intensities in tumor regions (compared images). Since all images are taken at optimal exposure times, they differ depending on the strength of signals. For a reliable comparison, the pictures are standardized to one exposure time, resulting in an illustration of differences in signal intensities. By manually drawing and modifying measurement regions, signal intensities can be quantified in intensity values. Once a measurement area is selected around the tumor, it can be cloned and moved to the next image to be compared with. Each region is calculated in pixels and mm² based on the current settings (stage height and binning). As a result, it gives information about the average signal, total signal, max. signal and average signal/exposure time (1/ms) within the created measurement area.

Another imaging technique which may be applied in connection with the present invention is the FMT technology (fluorescence molecular tomography), a laser based three-dimensional imaging system, which provides non-invasive, whole body, deep tissue imaging in small animal models and generates 3D reconstruction of fluorescence sources and/or allows measurement of fluorescence of fluorescence labeled analytes. The FMT technology is described, for example, in U.S. Pat. No. 6,615,063.

When performing the methods and uses of the invention, it is preferred that the fluorescence labeled potentially therapeutic antibody is to be administered to the subject prior to detecting the antibody, i.e., the signals from the fluorescence labeled antibody. Preferably, the antibody is administered parenterally, preferably intravenously to a subject. The term “administering” in all of its grammatical forms means administration of a panel of potentially therapeutic effective antibodies (preferably in the form of a pharmaceutical composition). The antibodies of said panel are either individually administered or as a panel, i.e., altogether, with individual administration being preferred.

If administered altogether, each antibody comprises preferably a different fluorescence label as described herein in order to identify each of the antibodies.

The term “antibody” refers to a monoclonal or a polyclonal antibody (see Harlow and Lane, “Antibodies, A Laboratory Manual”, CSH Press, Cold Spring Harbor, USA, 1988) which binds to a target, or a derivative of said antibody which retains or essentially retains its binding specificity. Preferred derivatives of such antibodies are chimeric antibodies comprising, for example, a mouse or rat variable region and a human constant region. The antibody may be in an isolated form. The term “functional fragment” as used herein refers to fragments of the antibodies as specified herein which retain or essentially retain the binding specificity of the antibodies like, separated light and heavy chains, Fab, Fab/c, Fv, Fab′, F(ab′)2. The term “antibody” also comprises bifunctional (bispecific) antibodies and antibody constructs, like single-chain Fvs (scFv) or antibody-fusion proteins. The term “scFv fragment” (single-chain Fv fragment) is well understood in the art and preferred due to its small size and the possibility to produce such fragments recombinantly. Said antibody or antibody binding portion is a human antibody or a humanized antibody. The term “humanized antibody” means, in accordance with the present invention, an antibody of non-human origin, where at least one complementarity determining region (CDR) in the variable regions such as the CDR3 and preferably all 6 CDRs have been replaced by CDRs of an antibody of human origin having a desired specificity. Optionally, the non-human constant region(s) of the antibody has/have been replaced by (a) constant region(s) of a human antibody. Methods for the production of humanized antibodies are described in, e.g., EP-A1 0 239 400 and WO90/07861. The term antibody or functional fragment thereof also includes heavy chain antibodies and the variable domains thereof, which are mentioned in WO 94/04678, WO 96/34103 and WO 97/49805, WO 04/062551, WO 04/041863, WO 04/041865, WO 04/041862 and WO 04/041867; as well as domain antibodies or “dAb's”, which are based on or derived from the heavy chain variable domain (VH) or the light chain variable domain (VL) of traditional 4 chain antibody molecules (see, e.g., Ward et al. 1989 Nature 341, 544-546).

An “isolated antibody” is one that has been identified and separated and/or recovered from a component of its natural environment. Contaminant components of its natural environment are materials that would interfere with diagnostic or therapeutic uses for the antibody, and may include enzymes, hormones, and other proteinaceous or non-proteinaceous solutes. Preferably, the antibody will be purified (1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or non-reducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step.

There are five classes of immunoglobulins: IgA, IgD, IgE, IgG, and IgM, having heavy chains designated a, 8, s, y and, respectively. The y and a classes are further divided into subclasses on the basis of relatively minor differences in CH sequence and function, e.g., humans express the following subclasses: IgG1, IgG2, IgG3, IgG4, IgA1, and IgA2.

The term “variable” refers to the fact that certain segments of the variable domains differ extensively in sequence among antibodies. The V domain mediates antigen binding and defines specificity of a particular antibody for its particular antigen. However, the variability is not evenly distributed across the 1-10-amino acid span of the variable domains. Instead, the V regions consist of relatively invariant stretches called framework regions (FRs) of 15-30 amino acids separated by shorter regions of extreme variability called “hypervariable regions” that are each 9-12 amino acids long. The variable domains of native heavy and light chains each comprise four FRs, largely adopting a P-sheet configuration, connected by three hypervariable regions, which form loops connecting, and in some cases forming part of, the P-sheet structure. The hypervariable regions in each chain are held together in close proximity by the FRs and, with the hypervariable regions from the other chain, contribute to the formation of the antigen-binding site of antibodies (see Kabat et al., Sequences of Proteins of Immunological Interest, 5th Ed. Public Health Service, National Institutes of Health, Bethesda, Md. (1991)). The constant domains are not involved directly in binding an antibody to an antigen, but exhibit various effector functions, such as participation of the antibody in antibody dependent cellular cytotoxicity (ADCC).

The term “hypervariable region” when used herein refers to the amino acid residues of an antibody which are responsible for antigen-binding. The hypervariable region generally comprises amino acid residues from a “complementarity determining region” or “CDR” (e.g. around about residues 24-34 (L1), 5056 (L2) and 89-97 (L3) in the VL, and around about 1-35 (H1), 50-65 (H2) and 95-102 (113) in the VH; Kabat et al., Sequences of Proteins of Immunological Interest, 5th Ed. Public Health Service, National Institutes of Health, Bethesda, Md. (1991)) and/or those residues from a “hypervariable loop” (e.g. residues 26-32 (L1), 50-52 (L2) and 91-96 (U) in the VL, and 26-32 (HI), 53-55 (1-12) and 96-101 (H3) in the VH; Chothia and Lesk J. Mol. Biol. 196: 901-917 (1987)).

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to polyclonal antibody preparations, which include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” is not to be construed as requiring production of the antibody by any particular method.

The monoclonal antibodies herein include “chimeric” antibodies in which a portion of the heavy and/or light chain is identical with or homologous to corresponding sequences in antibodies derived from a particular species or belonging to a particular antibody class or subclass, while the remainder of the chain (s) is identical with or homologous to corresponding sequences in antibodies derived from another species or belonging to another antibody class or subclass, as well as fragments of such antibodies, so long as they exhibit the desired biological activity (see U.S. Pat. No. 4,816,567; and Morrison et al., Proc. Natl. Acad. Sci. USA, 81: 6851-6855 (1984)). Chimeric antibodies of interest herein include “primatized” antibodies comprising variable domain antigen-binding sequences derived from a non-human primate (e.g. Old World Monkey, Ape etc), and human constant region sequences.

An “intact” antibody is one that comprises an antigen-binding site as well as a CL and at least heavy chain constant domains, CH1, CH2 and CH3. The constant domains may be native sequence constant domains (e.g. human native sequence constant domains) or amino acid sequence variant thereof. Preferably, the intact antibody has one or more effector functions.

An “antibody fragment” comprises a portion of an intact antibody, preferably the antigen binding or variable region of the intact antibody. Examples of antibody fragments include Fab, Fab′, F (ab′) 2, and Fv fragments; diabodies; linear antibodies (see U.S. Pat. No. 5,641,870, Example 2; Zapata et al., Protein Eng. 8 (10): 1057-1062 [1995]); single-chain antibody molecules; and multispecific antibodies formed from antibody fragments. Papain digestion of antibodies produces two identical antigen-binding fragments, called “Fab” fragments, and a residual “Fc” fragment, a designation reflecting the ability to crystallize readily. The Fab fragment consists of an entire L chain along with the variable region domain of the H chain (VH), and the first constant domain of one heavy chain (CH1). Each Fab fragment is monovalent with respect to antigen binding, i.e., it has a single antigen-binding site. Pepsin treatment of an antibody yields a single large F (ab′) 2 fragment which roughly corresponds to two disulfide linked Fab fragments having divalent antigen-binding activity and is still capable of cross-linking antigen. Fab′ fragments differ from Fab fragments by having additional few residues at the carboxy terminus of the CH1 domain including one or more cysteines from the antibody hinge region. Fab′-SH is the designation herein for Fab′ in which the cysteine residue (s) of the constant domains bear a free thiol group. F(ab′) 2 antibody fragments originally were produced as pairs of 8 Fab′ fragments which have hinge cysteines between them. Other chemical couplings of antibody fragments are also known.

The Fc fragment comprises the carboxy-terminal portions of both H chains held together by disulfides. The effector functions of antibodies are determined by sequences in the Fc region, which region is also the part recognized by Fc receptors (FcR) found on certain types of cells.

“Fv” is the minimum antibody fragment which contains a complete antigen-recognition and-binding site. This fragment consists of a dimer of one heavy-and one light-chain variable region domain in tight, non-covalent association. From the folding of these two domains emanate six hypervariable loops (3 loops each from the H and L chain) that contribute the amino acid residues for antigen binding and confer antigen binding specificity to the antibody. However, even a single variable domain (or half of an Fv comprising only three CDRs specific for an antigen) has the ability to recognize and bind antigen, although at a lower affinity than the entire binding site.

“Single-chain Fv” also abbreviated as “sFv” or “scFv” are antibody fragments that comprise the VH and VL antibody domains connected into a single polypeptide chain.

Preferably, the sFv polypeptide further comprises a polypeptide linker between the VH and VL domains which enables the sFv to form the desired structure for antigen binding. For a review of sFv, see Pluckthun in The Pharmacology of Monoclonal Antibodies, vol. 113, Rosenburg and Moore eds., Springer-Verlag, New York, pp. 269-315 (1994); Borrebaeck 1995, infra.

The term “diabodies” refers to small antibody fragments prepared by constructing sFv fragments (see preceding paragraph) with short linkers (about 5-10 residues) between the VH and VL domains such that inter-chain but not intra-chain pairing of the V domains is achieved, resulting in a bivalent fragment, i.e., fragment having two antigen-binding sites. Bispecific diabodies are heterodimers of two “crossover” sFv fragments in which the VH and VL domains of the two antibodies are present on different polypeptide chains. Diabodies are described more fully in, for example, EP 404,097; WO 93/11161; and Hollinger et al., Proc. Natl. Acad. Sci. USA, 90: 6444-6448 (1993).

“Humanized” forms of non-human (e.g., rodent) antibodies are chimeric antibodies that contain minimal sequence derived from the non-human antibody. For the most part, humanized antibodies are human immunoglobulins (recipient antibody) in which residues from a hypervariable region of the recipient are replaced by residues from a hypervariable region of a non-human species (donor antibody) such as mouse, rat, rabbit or non-human primate having the desired antibody specificity, affinity, and capability. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin. For further details, see Jones et al., Nature 321: 522-525 (1986); Riechmann et al., Nature 332: 323-329 (1988); and Presta, Curr. Op. Struct. Biol. 2: 593-596 (1992).

The antibody applied in the methods and uses of the present invention is labeled with a fluorescence label, preferably a fluorescence label which is detectable by near-infrared fluorescence imaging. Accordingly, when used in the context of the present invention, the term “fluorescence” includes fluorescence labels commonly known in the art or as described herein and also includes preferably near-infrared fluorescence labels, the latter being preferred in the context of the present invention.

A “fluorescent label” as used herein characterizes a molecule which comprises a fluorophore. A fluorophore, which is sometimes also termed fluorochrome, is a functional group in a molecule which will absorb energy of a specific wavelength and re-emit energy at a different wavelength. Said different wavelength, when compared to the said specific (predetermined) wavelength, is re-emitted with a wavelength which is distinguishable from the specific (predetermined) wavelength, for example it is re-emitted with a longer wavelength or with a shorter wave-length, however in the latter case with decreased intensity. The amount and wavelength of the emitted energy depends on both the fluorophore and the chemical environment of the fluorophore.

It is envisaged that the labeled antibody comprises more than one fluorescence or near-infrared fluorescence label, for example, 2, 3, 4, 5, 6, 7, 8, 9 or 10. Likewise, it is envisaged that 2, 3, 4, 5, 6, 7, 8, 9 or 10 different fluorescence or near-infrared fluorescence labeled antibodies are applied in the methods of the present invention.

Accordingly, it is envisaged that the fluorescent entity as used in the context of the present invention comprises just one sort of fluorescent labels or a mixture of at least two, three, four, five or even more different sorts of fluorescent labels. “Just one sort” means that the fluorescent labels contain one and the same fluorophore, while different sorts means that the different fluorescent labels comprise different fluorophores and therefore show different absorptions and/or emission characteristics. Typically the skilled person would use the same sort of fluorescent label when labeling a panel of antibodies that bind to the same desired target.

When just one sort of fluorescent label is used, typically also for all antibodies used in the above method the same labeling ratio is utilized. The labeling ratio as used herein means the number of label molecules per molecule antibody. In one embodiment the ratio of fluorescent label per antibody is 1.8-2.4:1. For example the ratio of anti-HER3 antibodies to Cy5-label was one antibody molecule per 2 Cy5 molecules (i.e. a labeling ratio of 1:2).

All antibodies in the method reported herein are used in the same concentration, i.e. in the same amount of injected antibody per bodyweight. The concentration is selected to sufficiently cover the antigen bearing tumor and thereby enabling the antibodies to be detected (e.g. in one case the amount of injected antibody is 10 mg per kg bodyweight for all antibodies of the panel, in another case the antibody concentration is 20 mg per kg bodyweight for all antibodies of the panel).

These “different” characteristics may be “unmixed” subsequently, e.g. by way of software aided evaluations. Means and methods to un-mix the emission of more than one different fluorophore are well known to the skilled person. The fluorescent label can be covalently and/or non-covalently linked to the spacer or to the second entity of the fluorescent analyte, using any suitable reactive group on the fluorescent label and a compatible functional group on the spacer or the second entity.

In the context of the present invention, said fluorescent label is preferably selected from the group comprising quantum dot agents, fluorescent proteins, fluorescent dyes, pH-sensitive fluorescent dyes, voltage sensitive fluorescent dyes and/or fluorescent labeled microspheres.

An example of a fluorescent label are “Quantum dot agents” or “Quantum dots”, also known as nanocrystals, are a special class of materials known as semiconductors, which are crystals composed of periodic groups of II-VI, III-V, or IV-VI materials.

Another example is a “Fluorescent protein” which includes for example green fluorescent protein (GFP), CFP, YFP, BFP either enhanced or not. Further fluorescent proteins are described in Zhang, Nat Rev Mol Cell Biol. 2002, 12, pages 906-18 or in Giepmans, Science. 2006, 312, pages 217-24.

“Fluorescent dyes” includes all kinds of fluorescent labels including but not limited to, Fluorescein including all its derivatives like for example FITC; Rhodamine including all its derivatives such as tetramethylrhodamine (TAMRA) and its isothiocyanate derivative (TRITC), sulforhodamine 101 (and its sulfonyl chloride form Texas Red), Rhodamine Red, and other derivatives of rhodamine which include newer fluorophores such as Alexa 546, Alexa 555, Alexa 633, DyLight 549 and DyLight 633); Alexa Fluors (the Alexa Fluor family of fluorescent dyes is produced by Molecular Probes); DyLight Fluor which is a family of fluorescent dyes are produced by Dyomics, ATTO Dyes, which represent a series of fluorescent labels and dyes manufactured by ATTO-TEC GmbH in Siegen, WO/2007/067978 Japan); LaJolla Blue (Diatron, Miami, Fla.); indocyanine green (ICG) and its analogs (Licha et al., 1996, SPIE 2927:192-198; Ito et al., U.S. Pat. No. 5,968,479); indotricarbocyanine (ITC; WO 98/47538), and chelated lanthanide compounds. Fluorescent lanthanide metals include europium and terbium.

An antibody is preferably labeled with a fluorescence label detectable by NIRF, i.e. it is preferably labeled with a near-infrared (NIR) fluorescence label. NIR fluorescence labels with excitation and emission wavelengths in the near infrared spectrum are used, i.e., 640-1300 nm preferably 640-1200 nm, and more preferably 640-900 nm. Use of this portion of the electromagnetic spectrum maximizes tissue penetration and minimizes absorption by physiologically abundant absorbers such as hemoglobin (<650 nm) and water (>1200 nm). Ideal near infrared fluorochromes for in vivo use exhibit:

(1) narrow spectral characteristics,
(2) high sensitivity (quantum yield),
(3) biocompatibility,
(4) decoupled absorption and excitation spectra, and
(5) photo stability.

Various near infrared (NIR) fluorescence labels are commercially available and can be used to prepare a fluorescent entity according to this invention. Exemplary NIRF labels include the following: Cy5.5, Cy5 and Cy7 (Amersham, Arlington Hts., IL; IRD41 and IRD700 (L1-COR, Lincoln, Nebr.); NIR-I, (Dejindo, Kumamoto, Japan); LaJolla Blue (Diatron, Miami, Fla.); indocyanine green (ICG) and its analogs (Licha, K., et al., SPIE—The International Society for Optical Engineering 1996; Vol. 2927: 192-198; U.S. Pat. No. 5,968,479); indotricarbocyanine (ITC; WO 98/47538); and chelated lanthanide compounds and SF64, 5-29, 5-36 and 5-41 (from WO 2006/072580). Fluorescent lanthanide metals include europium and terbium. Fluorescence properties of lanthanides are described in Lackowicz, J. R., Principles of Fluorescence Spectroscopy, 2nd Ed., Kluwer Academic, New York, (1999).

“Fluorescent microspheres” are described in great detail in WO/2007/067978 which is incorporated herein by reference.

In a further embodiment of the present invention, at least one fluorescent label of the fluorescent entity is activatable. It is also envisaged that the fluorescent entity is activatable.

As mentioned before, it is known that the amount and wavelength of the energy emitted by a fluorescent dye depends on both the fluorophore and the chemical environment of the fluorophore. It follows that fluorescent dyes may react pH-sensitive or voltage sensitive, i.e. they are activatable by such changes in the chemical environment. Further activatable fluorescent labels are described for example in great detail in US 2006/0147378 A1, U.S. Pat. No. 6,592,847, U.S. Pat. No. 6,083,486, WO/2002/056670 or US 2003/0044353 A1, all of which are incorporated herein by reference.

By “activation” of a fluorescent label/entity is meant any change to the label/entity that alters a detectable property, e.g., an optical property, of the label/entity. This includes, but is not limited to, any modification, alteration, or binding (covalent or non-covalent) of the label/entity that results in a detectable difference in properties, e.g., optical properties e.g., changes in the fluorescence signal amplitude (e.g., dequenching and quenching), change in wavelength, fluorescence lifetime, spectral properties, or polarity. Optical properties include wavelengths, for example, in the visible, ultraviolet, near-infrared, and infrared regions of the electromagnetic spectrum. Activation can be, without limitation, by enzymatic cleavage, enzymatic conversion, phosphorylation or dephosphorylation, conformation change due to binding, enzyme-mediated splicing, enzyme-mediated transfer of the fluorophore, hybridization of complementary DNA or RNA, analyte binding such as association with an analyte such as Na+, K+, Ca2+, Cl−, or another analyte, change in hydrophobicity of the probe environment, and chemical modification of the fluorophore. Activation of the optical properties may or may not be accompanied by alterations in other detectable properties, such as (but not limited to) magnetic relaxation and bioluminescence.

Fluorescence labeling is accomplished using a chemically reactive derivative of a fluorophore. Common reactive groups include amine reactive isothiocyanate derivatives such as FITC and TRITC (derivatives of fluorescein and rhodamine), amine reactive succinimidyl esters such as NHS-fluorescein, and sulfhydryl reactive maleimide activated fluors such as fluorescein-5-maleimide. Reaction of any of these reactive dyes with another molecule results in a stable covalent bond formed between a fluorophore and a labelled molecule. Accordingly, the antibody of the present invention can be labeled with the aforementioned fluorescence labels. Other suitable fluorescence labels envisaged by the present invention are Alexa Fluors, Dylight fluors and ATTO Dyes. Likewise, fluoresecent proteins such as GFP, YFP or CFP may be used. Also, activatable fluorescent dyes which are activated by pH changes or voltage, temperature are envisaged to be used. It is also envisaged that the labeled antibody comprises more than one fluorescence or near-infrared fluorescence label, for example, 2, 3, 4, 5, 6, 7, 8, 9 or 10. Likewise, it is envisaged that 2, 3, 4, 5, 6, 7, 8, 9 or 10 different fluorescence or near-infrared fluorescence labeled antibodies are applied in the kits, methods and uses of the invention.

In the alternative, the antibody applied in the methods of the present invention may be indirectly labeled. For example, an unlabeled antibody is labeled (due to its binding to the constant region of the unlabeled antibody) by a second antibody comprising a fluorescence label, wherein said second antibody is directed against the unlabeled antibody.

In said alternative, the fluorescent label of the second antibody is activated once the epitope binding domain of said second antibody has bound to its target.

“Activated” includes the activation of activatable fluorescent labels which have been mentioned herein before. “Activated” also includes “FRET-based” effects. Förster resonance energy transfer (abbreviated FRET), also known as fluorescence resonance energy transfer, resonance energy transfer (RET) or electronic energy transfer (EET), is a mechanism describing energy transfer between two fluorophores. FRET provides an indication of proximity between donor and acceptor fluorophores. When a donor is excited with incident radiation at a defined frequency, some of the energy that the donor would normally emit as fluorescence is transferred to the acceptor, when the acceptor is in sufficiently close proximity to the donor (typically, within about 50 Angstroms for most donor fluorophores). At least some of the energy transferred to the acceptor is emitted as radiation at the fluorescence frequency of the acceptor. FRET is further described in various sources, such as “FRET Imaging” (Jares-Erijman, E. A, and Jovin, T. M, Nature Biotechnology, 21(11), (2003), pg 1387-1395), which is incorporated herein by reference for all purposes. Screening systems based on such FRET effects are well known and described for example in WO 2006107864 which is included herein in its entirety.

Methods for coupling fluorescent labels including NIR fluorescence labels are well known in the art. The conjugation techniques of these labels to an antibody have significantly matured during the past years and an excellent overview is given in Aslam, M., and Dent, A., Bioconjugation (1998) 216-363, London, and in the chapter “Macromolecule conjugation” in Tijssen, P., “Practice and theory of enzyme immunoassays” (1990), Elsevier, Amsterdam.

Appropriate coupling chemistries are known from the above cited literature (Aslam, supra). The fluorescent label, depending on which coupling moiety is present, can be reacted directly with the antibody either in an aqueous or an organic medium. The coupling moiety is a reactive group or activated group which is used for chemically coupling of the fluorochrome label to the antibody. The fluorochrome label can be either directly attached to the antibody or connected to the antibody via a spacer to form a NIR fluorescence label conjugate comprising the antibody and a NIR fluorescence label. The spacer used may be chosen or designed so as to have a suitably long in vivo persistence (half-life) inherently.

The desired target on the surface of a mammalian cell refers to the target antigen on said cell surface, which can comprise several epitopes. The term “epitope” denotes a protein determinant of the target antigen on the cell surface of the mammalian cell capable of specifically binding to an antibody. Epitopes usually consist of chemically active surface groupings of molecules such as amino acids or sugar side chains and usually epitopes have specific three dimensional structural characteristics, as well as specific charge characteristics. Conformational and nonconformational epitopes are distinguished in that the binding to the former but not the latter is lost in the presence of denaturing solvents. Typically, an epitope comprises 3, 4, 5, 6, 7, 8, 9 or 10 amino acids. The “surface” of a cell includes the cell membrane (also called the plasma membrane). It is the biological membrane separating the interior of a cell (preferably a mammalian cell) from the outside environment. The cell membrane surrounds all cells and it is semi-permeable, controlling the movement of substances in and out of cells. It contains a wide variety of biological molecules, primarily lipids and proteins. These proteins include transmembrane proteins, lipid anchored proteins and peripheral proteins which are preferred targets in the sense of the present invention.

“Transmembrane proteins” span the membrane and have a hydrophilic cytosolic domain, which interacts with internal molecules, a hydrophobic membrane-spanning domain that anchors it within the cell membrane, and a hydrophilic extracellular domain that interacts with external molecules. “Lipid anchored proteins” are covalently-bound to single or multiple lipid molecules; hydrophobically insert into the cell membrane and anchor the protein. “Peripheral proteins” are attached to integral membrane proteins, or associated with peripheral regions of the lipid bilayer.

In accordance with the teaching of the present invention, proteins which extend to the exterior of a cell such as receptor-molecules are typically available as an antigen that can be targeted by antibodies. These proteins are preferred.

The term “receptor-molecule” relates thus to a protein on the cell membrane or within the cytoplasm or cell nucleus that binds to a ligand and typically transduces a signal, such as metabotropic receptors, G protein-coupled receptors, guanylyl cyclise receptors, receptor tyrosine kinases, muscarinic acetylcholine receptors, adenosine receptors, adrenoceptors, GABA receptors, angiotensin receptors, cannabinoid receptors, cholecystokinin receptors, dopamine receptors, glucagon receptors, metabotropic glutamate receptors, histamine receptors, olfactory receptors, opioid receptors, chemokine receptors including cytokine receptors, calcium-sensing receptor, somatostatin receptors, serotonin receptors, secretin receptors, ionotropic receptors, or Fc receptors. Further preferred targets are CA 15-3, CA 19-9, CA 72-4, CFA, MUC-1, MAGE, p53, ETA, CA-125, CEA, AFP, PSA, PSMA, CA IX and/or OE8.

In a further aspect, the present invention relates to the use of the method of the present invention for selecting in vivo an antibody against a desired target on the surface of a mammalian cell that is a candidate for the therapeutically most effective antibody out of a panel of potentially therapeutic effective antibodies.

In a still further aspect, the present invention relates to the use of near-infrared fluorescence imaging (NIRF) in a method for in vivo selecting an antibody against a desired target on the surface of a mammalian cell that is a candidate for the therapeutically most effective antibody out of a panel of potentially therapeutic effective antibodies comprising
(a) detecting each of the fluorescence labeled potentially therapeutic effective antibodies of said panel after binding to its target in a subject by near-infrared fluorescence imaging (NIRF); and
(b) selecting the antibody which shows after binding to its target the highest fluorescence signals.

The embodiments described in the context of the methods of the present invention are applicable in the context of this use, mutatis mutandis.

Also contemplated by the present invention is an antibody selected by the methods the present invention. Said antibody is preferably for use in the treatment of cancer.

Likewise, the present invention contemplates the use of an antibody selected by the methods of the present invention for the preparation of a pharmaceutical composition for the treatment of cancer. Said pharmaceutical composition may optionally comprise a carrier.

“Carriers” as used herein include pharmaceutically acceptable carriers, excipients, or stabilizers which are nontoxic to the cell or mammal being exposed thereto at the dosages and concentrations employed. Often the physiologically acceptable carrier is an aqueous pH buffered solution.

Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN, polyethylene glycol (PEG), and PLURONICS™.

Pharmaceutical formulations of the antibodies used in accordance with the present invention are prepared for storage by mixing an antibody having the desired degree of purity with optional pharmaceutically acceptable carriers, excipients or stabilizers (Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed, and include buffers such as acetate, Tris, phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol, and mcresol); low molecular weight (less than about 10 residues) polypeptides; proteins, such as serum albumin, gelatin, or immunoglobulins; hydrophilic polymers such as polyvinylpyllolidone; amino acids such as glycine, glutamine, asparagine, histidine, arginine, or lysine; monosaccharides, disaccharides, and other carbohydrates including glucose, mannose, or dextrins; chelating agents such as EDTA; tonicifiers such as trehalose and sodium chloride; sugars such as sucrose, mannitol, trehalose or sorbitol; surfactant such as polysorbate; salt-forming counter-ions such as sodium; metal complexes (e.g. Zn-protein complexes); and/or non-ionic surfactants such as TWEEN™, PLURONICS™ or polyethylene glycol (PEG). The antibody preferably comprises the antibody at a concentration of between 5-200 mg/ml, preferably between 10-100 mg/ml.

The active ingredients may also be entrapped in microcapsules prepared, for example, by coacervation techniques or by interfacial polymerization, for example, hydroxymethylcellulose or gelatinmicrocapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

The formulations to be used for in vivo administration must be sterile. This is readily accomplished by filtration through sterile filtration membranes.

Another aspect of the present invention is a kit for use in any of the methods of the present invention comprising a panel of potentially therapeutic effective fluorescence labeled antibodies and means for near-infrared fluorescence imaging to detect said antibody in a subject.

The kit may contain a package insert. The term “package insert” is used to refer to instructions customarily included in commercial packages of therapeutic products, that contain information about the indications, usage, dosage, administration, contraindications and/or warnings concerning the use of such therapeutic products.

The kit comprises a container and a composition contained therein comprising an antibody selected in accordance with the methods of the present invention. The kit may also comprise a label or package insert on or associated with the container. Suitable containers include, for example, bottles, vials, syringes, etc. The containers may be formed from a variety of materials such as glass or plastic. The label or package insert indicates that the composition is used for treating cancer as described herein, more specifically breast cancer. The label or package insert may further comprise instructions for administering the antibody composition to a cancer patient. Additionally, the kit may further comprise a second container comprising a substance which detects the antibody of this invention, e.g., a second antibody which binds to the antibodies of this invention. The substance may be labeled with a detectable label such as those disclosed herein. The second container may contain e.g., a pharmaceutically-acceptable buffer, such as bacteriostatic water for injection (BWFI), phosphate-buffered saline, Ringer's solution and dextrose solution. The kit may further include other materials desirable from a commercial and user standpoint, including other buffers, diluents, filters, needles, and syringes. The label or package insert may provide a description of the composition as well as instructions for the intended in vivo or diagnostic use.

### FIGURES

The Figures show:

FIG. 1: Correlation of NIRF signal intensities in vivo (1a, 1b, 1c) with formalin fixed/paraffin embedded hematoxylin tissue slides (2a, 2b, 2c) and multispectral imaging of explanted tumor tissue (3a, 3b, 3c) using Trastuzumab-Cy5 (a), Pertuzumab-Cy5 (b) and Xolair-Cy5 (c).

FIG. 2: In vivo imaging of potentially therapeutic antibodies (MOR6395_MM046, MOR6396_MM056, MOR6420_MM048, MOR6434_MM047, MOR6971_M M044) directed against Cyr61.

FIG. 3: Ex vivo imaging of potentially therapeutic antibodies (MOR6395_MM046, MOR6396_MM056, MOR6420_MM048, MOR6434_MM047, MOR6971_M M044) directed against Cyr61.

FIG. 4: In vivo efficacy of potentially therapeutic antibodies (MOR6395_MM046, MOR6396_MM056, MOR6420_MM048, MOR6434_MM047, MOR6971_M M044) directed against Cyr61.

FIG. 5: Diagram of fluorescence intensities of in vivo imaging of potentially therapeutic antibodies clone 1-clone 5 directed against HER3.

FIG. 6: In vivo efficacy of potentially therapeutic antibodies clone 1-clone 5 directed against HER3 in CALU-3 xenografts.

### EXAMPLES

The following examples illustrate the invention. These examples should not be construed as to limit the scope of this invention. The examples are included for purposes of illustration and the present invention is limited only by the claims.

### Example 1

In this example the utility of near-infrared fluorescence imaging to verify target expression and binding of antibodies to tumor tissue is demonstrated.

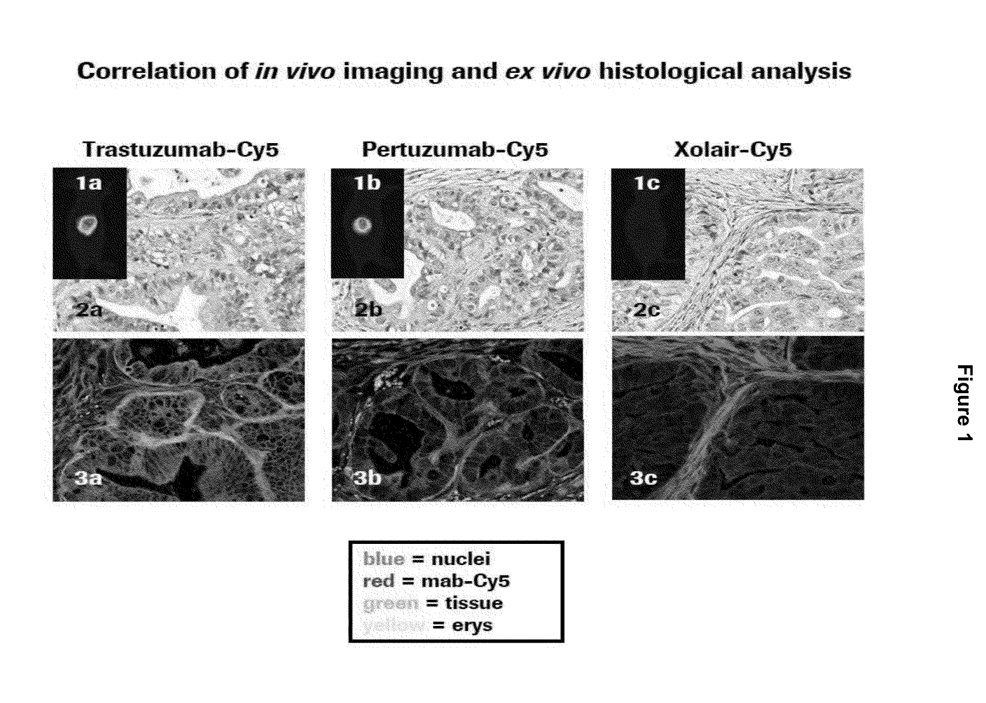

The therapeutic antibodies Trastuzumab, Pertuzumab and Xolair are labelled with an organic fluorophore (Cy5) and injected i.v. in human lung xenografts. After a single i.v. injection of 50 microgram of Trastuzumab-Cy5 and Pertuzumab-Cy5 in tumor bearing mice, a strong fluorescence signal is detectable in the Her2 overexpressing tumor area after 24 to 48 hours. Subsequent analysis of explanted tumor tissue reveals that the Her2 specific antibodies bind only to tumor cells, but not to murine tissue. In contrast, no fluorescence signal is generated with the Cy5 labelled control antibody Xolair which is directed to human IgE (FIG. 1).

### Example 2

In this example the utility of near-infrared fluorescence imaging to verify the binding efficacy of different antibodies to one tumor associated antigen is demonstrated. The in vivo and ex vivo imaging results are confirmed by a preclinical study.

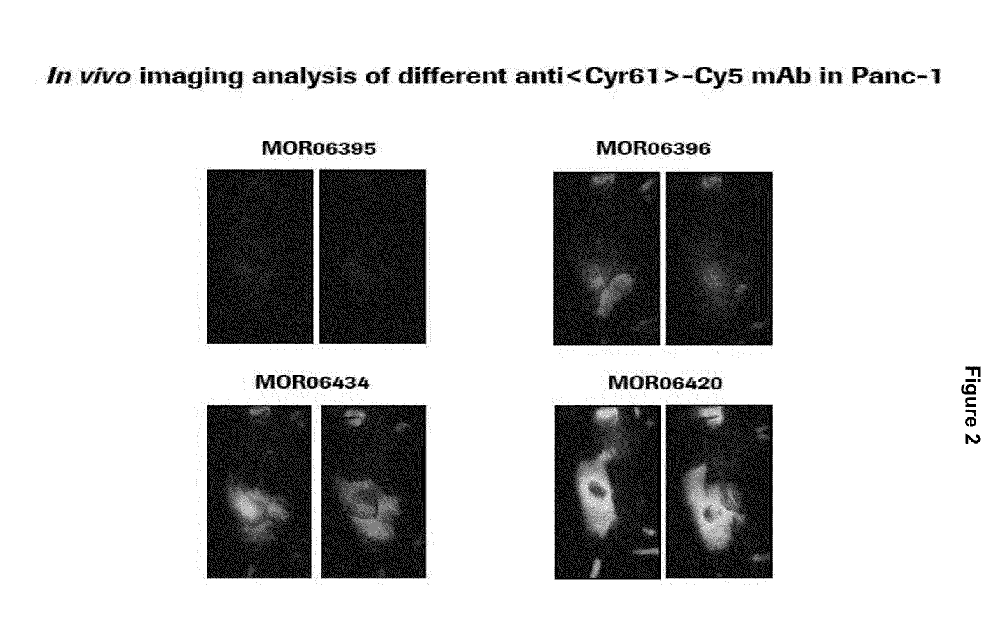

Four different therapeutic antibodies (MOR06395, MOR06396, MOR06434, MOR06420) against the tumor antigen Cyr61 are labelled with the organic fluorophore Cy5. Each of the labelled antibodies is injected in three s.c. tumor bearing SCID-beige mice (human pancreatic xenograft). After a single i.v. injection of 50 microgram of the labelled anti-Cyr61 mAb, the fluorescence signal is imaged in the tumor area after 24 to 48 hours (FIG. 2). The signals of all in vivo images are normalized, converted into pseudo-color and the intensities in the tumor area are analysed. Comparing the signal intensities of the four different antibodies with each other, the mAb MOR06420 show the highest signal intensity in the tumor area.

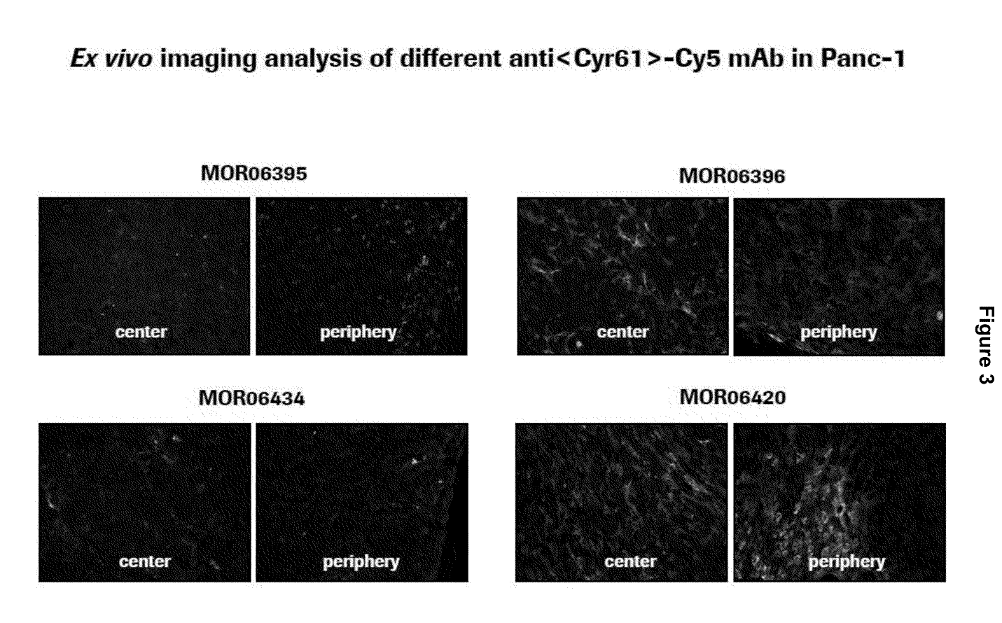

The results from in vivo imaging are confirmed by histological ex vivo analysis. For this, the tumors are explanted, fixed in formalin and embedded in paraffin. In the sliced and DAPI stained tumor tissue, the antibody MOR06420 show also the brightest signal intensity (FIG. 3).

From the in vivo and ex vivo results, the mAb MOR06420 can be prioritized as the antibody with the best binding efficacy. Therefore, the antibody MOR06420 should show the best therapeutic efficacy.

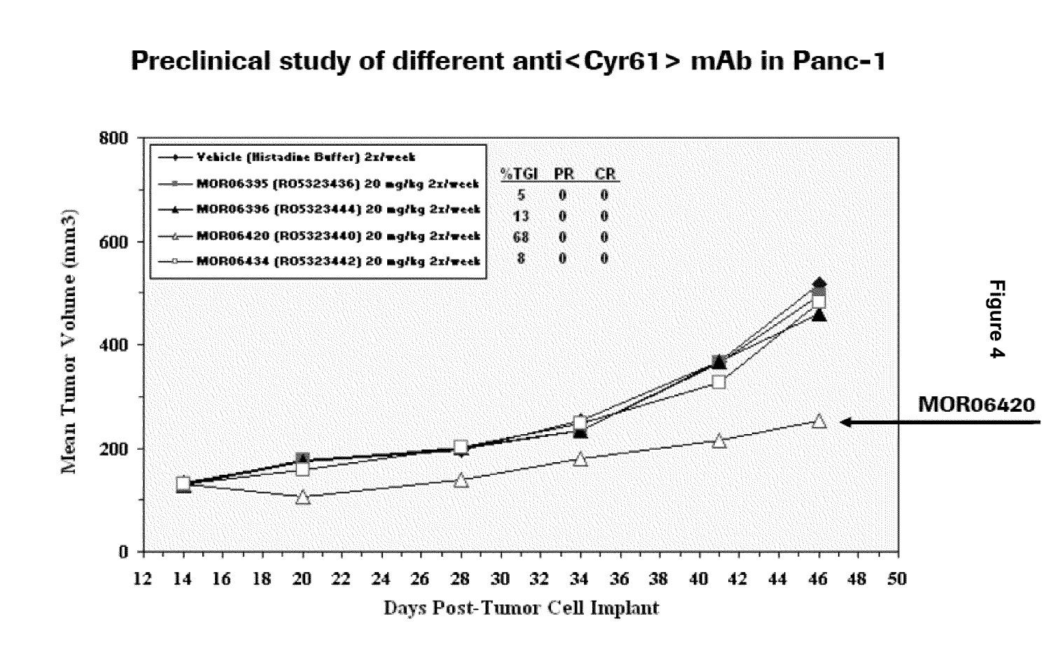

To confirm this hypothesis, the four different therapeutic antibodies against Cyr61 are tested in a preclinical study. The s.c. Panc-1 bearing SCID-beige mice are divided in five groups with ten mice each. The untreated vehicle group are injected i.p. with Histidine buffer twice a week. Each of the four treatment groups are injected i.p. with an anti-Cyr61 antibody concentration of 20 mg/kg twice a week. The tumor volume of all animals are measured over the time by calliper (FIG. 4).

The anti-Cyr61 monoclonal antibody MOR06420 appeared to possess the most potent antitumor activity in Panc-1 xenografts, with a tumor growth inhibition of 68%.

### Example 3

In this example the utility of near-infrared fluorescence imaging to verify the binding efficacy of different antibodies to one tumor associated antigen is demonstrated. The in vivo imaging results are confirmed by a preclinical study.

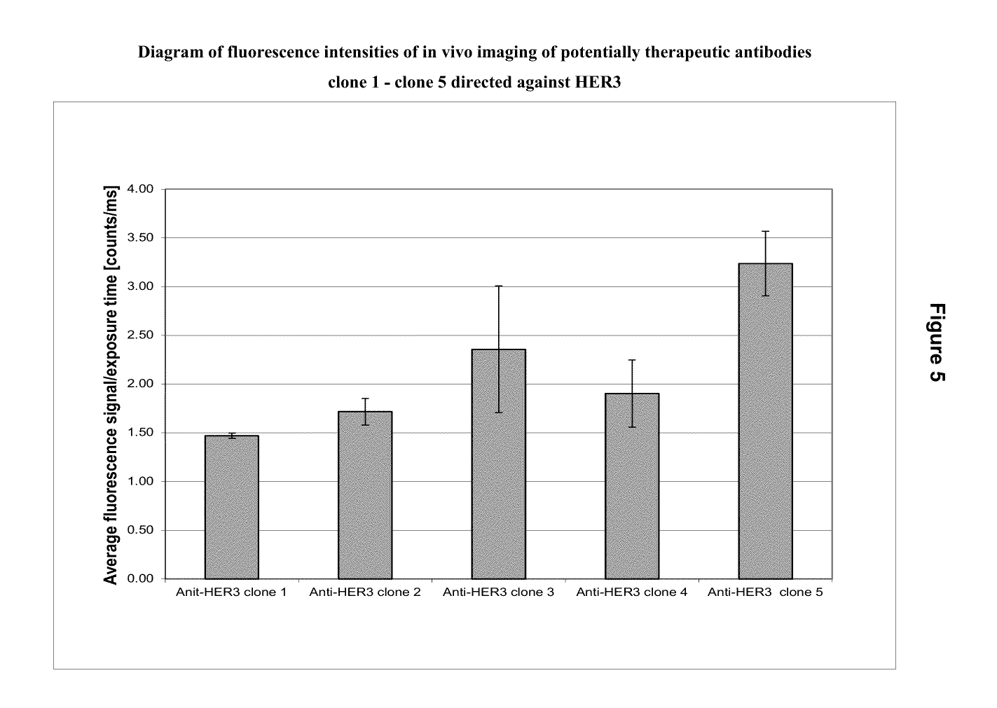

Five different therapeutic antibodies (clone 1 to clone 5) against the tumor antigen HER3 are labelled with the organic fluorophore Cy5. Each of the labelled antibodies is injected in three s.c. tumor bearing SCID hairless outbred mice (CALU-3 xenograft). After a single i.v. injection of 50 microgram of the labelled anti-HER3 mAb, the fluorescence signal is imaged in the tumor area after 24 to 48 hours. The signals of all in vivo images are normalized, converted into pseudo-color and the intensities in the tumor area are analyzed. Comparing the signal intensities of the five different antibodies (clone 1 to clone 5) with each other, the anti-HER3 mAb clone 5 shows the highest signal intensity in the tumor area (FIG. 5).

From the in vivo results anti-HER3 mAb clone 5 can be prioritized as the antibody with the best binding efficacy. Therefore, the anti-HER3 mAb clone 5 should show the best therapeutic efficacy.

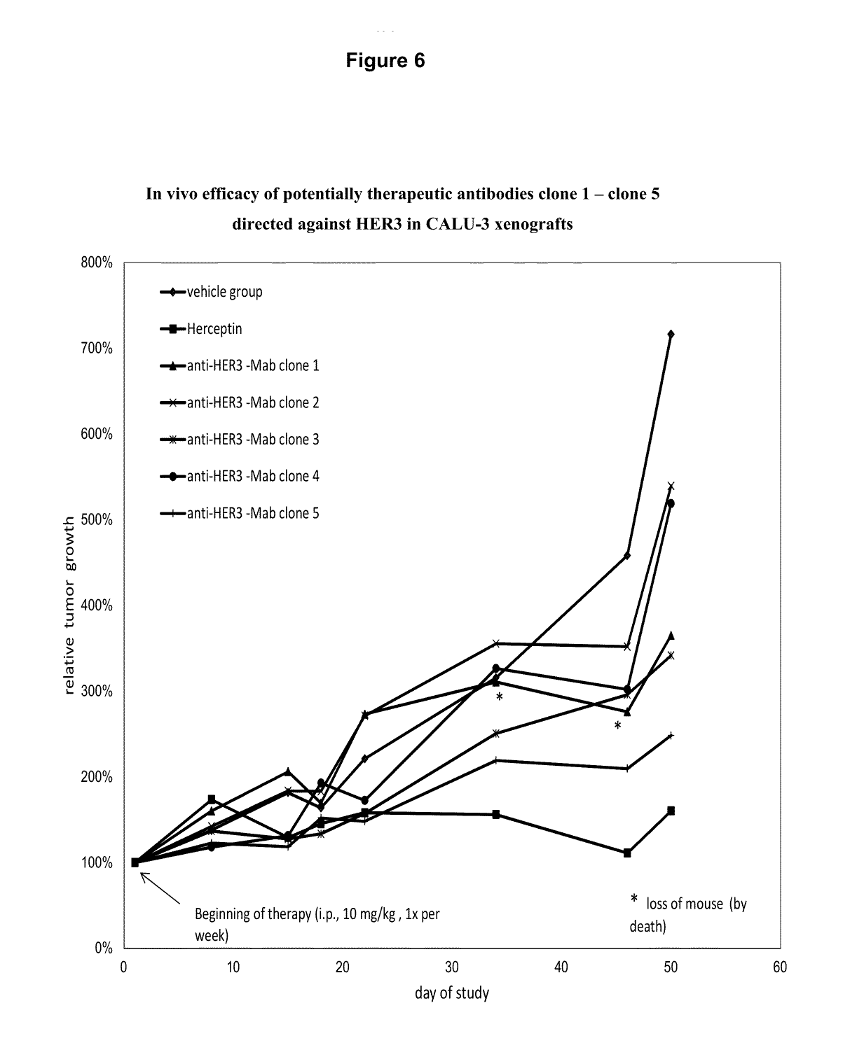

To confirm this hypothesis, the five different therapeutic antibodies against HER3 are tested in a preclinical study. The s.c. CALU-3 bearing SCID hairless outbred mice are divided in seven groups with eight mice each. The untreated vehicle group is injected i.p. with Histidine buffer once a week. Each of the five treatment groups are injected i.p. with an anti-HER3 antibody concentration of 10 mg/kg once a week. In two additional treatment groups, Herceptin or a Histidine buffer is injected i.p. once a week as a positive or negative control, respectively. The tumor volumes of all animals are measured over the time by caliper (FIG. 6).

The anti-HER3 monoclonal antibody clone 5 appeared to possess the most potent antitumor activity in CALU-3 xenografts.

1. A non-invasive method of in vivo selecting an antibody against a desired target on the surface of a mammalian cell of a subject that is a candidate for the therapeutically most effective antibody out of a panel of potentially therapeutic effective antibodies binding the same desired target each of which is fluorescence labeled comprising
(a) detecting each of the fluorescence labeled potentially therapeutic effective antibodies of said panel after binding to its target in a subject by near-infrared fluorescence imaging (NIRF); and
(b) selecting the antibody which shows after binding to its target the highest fluorescence signals.

2. The method of claim 1, wherein said panel of antibodies binding the same desired target is fluorescence labeled with just one sort of fluorescent label.

3. The method of any of the preceding claims, wherein said mammalian cell is a malignant cell.

4. The method of any of the preceding claims, wherein said panel of potentially therapeutic effective antibodies comprises antibodies intended to be used in therapy of said subject.

5. The method of any of the preceding claims, wherein the subject is to be administered the fluorescence labeled potentially therapeutic antibody prior to detecting said antibody.

6. The method of any of the preceding claims, wherein said subject is a mammal.

7. The method of any of the preceding claims, wherein said subject comprises a tumor which comprises malignant/cancerous cells having said desired target on their surface.

8. The method of claim 7, wherein said tumor is a xenograft, preferably a human xenograft.

9. The method of any of the preceding claims, wherein the target is a protein having an extracellular portion.

10. The method of any of the preceding claims, wherein near-infrared fluorescence imaging includes fluorescence reflectance imaging (FRI) or fluorescence-mediated tomography (FMT).

11. The method of any of the preceding claims which is for in vivo selecting an antibody which is most appropriate for therapy.

12. Use of the method of any of the preceding claims for in vivo selecting an antibody against a desired target on the surface of a mammalian cell that is a candidate for the therapeutically most effective antibody out of a panel of potentially therapeutic effective antibodies.

13. Use of near-infrared fluorescence imaging (NIRF) in a method for in vivo selecting an antibody against a desired target on the surface of a mammalian cell that is a candidate for the therapeutically most effective antibody out of a panel of potentially therapeutic effective antibodies binding the same desired target each of which is fluorescence labeled comprising
(a) detecting each of the fluorescence labeled potentially therapeutic effective antibodies of said panel after binding to its target in a subject by near-infrared fluorescence imaging (NIRF),
(b) selecting the antibody which shows after binding to its target the highest fluorescence signals.

14. Use of claim 13, wherein said panel of antibodies binding the same desired target is fluorescence labeled with just one sort of fluorescent label.

15. An antibody selected by the method of any of the preceding claims for use in the treatment of cancer.

16. Use of an antibody selected by the method of any of the preceding claims for the preparation of a pharmaceutical composition for the treatment of cancer.

17. A kit for use in any of the methods defined in the preceding claims comprising a panel of potentially therapeutic effective fluorescence labeled antibodies and means for near-infrared fluorescence imaging to detect said antibody in a subject.
